SEQUENCE LISTING (15 sequences)

SEQ ID NO: 1 - protein, 510 aa, ARTIFICIAL SEQUENCE
  mouse sequence
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - protein, 381 aa, ARTIFICIAL SEQUENCE
  truncated mouse sequence
NSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 3 - protein, 380 aa, ARTIFICIAL SEQUENCE
  Bovine truncated
DYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 4 - protein, 380 aa, ARTIFICIAL SEQUENCE
  Human truncated
DYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 5 - protein, 376 aa, ARTIFICIAL SEQUENCE
  Chicken 1 truncated
QYPTLKTPESEVSSFTASKVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 6 - protein, 387 aa, ARTIFICIAL SEQUENCE
  Possum truncated
SANPDPTAGTLNILPPTTKTISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 7 - protein, 381 aa, ARTIFICIAL SEQUENCE
  New truncated shrew
DCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 8 - protein, 387 aa, ARTIFICIAL SEQUENCE
  Python truncated
EKYQLPEDEDRSVTSDLDRDSISEYACQRRTTLKNYNQKFTDAFEILAENYEFNENKGFCTAFRRAASVLKCLPFTIVQVHDIEGVPWMGKQVKGIIEDIIEEGESSKVKAVLDNENYRSVKLFTSVFGVGLKTSDKWYRMGLRTLEEVKRDKNLKLTRMQKAGFLHYDDLTSCVSKAEADAASLIVQDVVWKIVPNAIVTIAGGFRRGKQTGHDVDFLITVPGSKQEEEELLHTVIDIWKKQELLLYYDLIESTFEDTKLPSRKVDALDHFQKCFAILKVHKEREDKGNSIRSKAFSEEEIKDWKAIRVDLVVVPFEQYAFALLGWTGSTQFERDLRRYATHEKKMMLDNHALYDKTKKIFLNAASEEEIFAHLGLDYLEPWERNA

SEQ ID NO: 9 - protein, 381 aa, ARTIFICIAL SEQUENCE
  truncated dog
DYTASPNPELQKTLPVAVKKISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 10 - protein, 382 aa, ARTIFICIAL SEQUENCE
  TRUNC MOLE
GDCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 11 - protein, 379 aa, ARTIFICIAL SEQUENCE
  Pika trunk
EYSANPSPGPQATPAVYKISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 12 - protein, 384 aa, ARTIFICIAL SEQUENCE
  TRUNC HEDGEHOG
DASFGSNPGSQNTPPLAIKKISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 13 - protein, 381 aa, ARTIFICIAL SEQUENCE
  truncated tree shrew
DHSTSPSPGPQKTPALAVQKISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 14 - protein, 394 aa, ARTIFICIAL SEQUENCE
  TRUNCATED PLATYPUS
LTNSAPINCMTETPSLATKQVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 15 - protein, 384 aa, ARTIFICIAL SEQUENCE
  TRUNCATED JERBOA
SSELELLDVSWLIECMGAGKPVEMTGRHQLVKQTFCLPGFILQDAFDILAENCEFRENEASCVEFMRAASVLKSLPFPIISVKDTEGIPWLGGKVKCVIEEIIEDGESSEVKALLNDERYKSFKLFTSVFGVGLKTAERWFRMGFRTLSTVKLDKSLTFTRMQKAGFLHYEDLVSCVTRAEAEAVSVLVQQAVVAFLPDALVSMTGGFRRGKKIGHDVDFLITSPEATEEEEQQLLHKVTNFWEQKGLLLYCDHVESTFEKCKLPSRKVDALDHFQKCFLILKLYRERVDSVKSSQQEGKGWKAIRVDLVMCPYECRAFALLGWTGSRQFERDLRRYATHERKMRLDNHALYDKTKRVFLKAESEEEIFAHLGLEYIEPLERNA

(19) United States
(12) Patent Application Publication
(10) Pub. No.: US 2024/0052391 A1
(43) Pub. Date: Feb. 15, 2024

(54) Enzymatic Synthesis of Polynucleotide Probes

(71) Applicant: DNA Script, Le Kremlin-Bicêtre (FR)

(72) Inventors: Xavier Godron, Paris (FR); Supaporn Niyomchon, Paris (FR); Elise Champion, Paris (FR)

(21) Appl. No.: 18/034,487

(22) PCT Filed: Oct. 27, 2021
(86) PCT No.: PCT/EP2021/079847

§ 371 Date: Apr. 28, 2023

(30) Foreign Application Priority Data
Oct. 29, 2020 (EP) 20306298

(51) Int. Cl. C12P 19/34; C12N 9/16

(52) U.S. Cl. CPC C12P 19/34; C12N 9/16; C12Y 301/21007

(57) ABSTRACT

The present invention is directed to methods and kits for enzymatic synthesis of labeled polynucleotide probes using template-free DNA polymerases.

### BACKGROUND

Interest in enzymatic approaches to polynucleotide synthesis has recently increased both because of increased demand for synthetic polynucleotides in many areas, such as synthetic biology, CRISPR-Cas9 applications, high-throughput sequencing, and the like, and because of the limitations of chemical approaches to polynucleotide synthesis, such as the difficulty of performing multi-step synthesis reactions under inert atmospheres and moisture-free environments, the upper limits on product length, the use of, and needed disposal of, organic solvents, and so on, e.g., Jensen et al, Biochemistry, 57: 1821-1832 (2018). Currently, most enzymatic approaches employ a template-free polymerase to repeatedly add 3′-O-blocked nucleoside triphosphates to a single stranded initiator or an elongated strand attached to a support followed by deblocking until a polynucleotide of the desired sequence is obtained. In such enzymatic approaches to polynucleotide synthesis, enzyme specificity often presents a “double-edged sword” in that same specificity that was engineered for usual monomers may prevent or greatly reduce activity when it is desired to incorporate unusual monomers with labels or other adducts.

In view of the above, enzymatic synthesis of polynucleotides would be advanced if methods were available for high efficiency incorporation of various labels or other adducts into enzymatically synthesized nucleic acid probes.

### SUMMARY OF THE INVENTION

The present invention is directed to methods, kits and compositions for template-free enzymatic synthesis of polynucleotide probes having predetermined sequences and a plurality of attached fluorescent dyes and/or quencher molecules.

In one aspect, methods of the invention include a method of synthesizing a polynucleotide probe having a predetermined sequence and a plurality of labels with the following steps: (a) providing an initiator having a 3′-penultimate deoxyinosine and a 3′-terminal nucleotide having a free 3′-hydroxyl; (b) repeating elongation cycles comprising steps of: (i) contacting under elongation conditions the initiator or elongated fragments having free 3′-O-hydroxyls with a 3′-O-blocked nucleoside triphosphate and a template-independent DNA polymerase so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, until the polynucleotide of the polynucleotide probe is formed, wherein (A) at least one of the 3′-O-blocked nucleoside triphosphate is a 3′-O-blocked-(alkyne/azide)-nucleoside triphosphate or a 3′-O-blocked-(protected alkyne/protected azide)-nucleoside triphosphate , (B) in a final elongation cycle a nucleoside triphosphate having a 3′-alternative blocking group or 3′-O-alternative blocking group is contacted with said elongated fragments having free 3′-hydroxyls, (C) the alternative blocking group has a reactive moiety and may be the same or different than the blocking group, and (D) said step of deblocking is omitted; (c) reacting a first label having an (alkyne/azide) group with the at least one incorporated 3′-blocked-(alkyne/azide)-nucleoside triphosphate; (d) reacting the reactive moiety of the 3′-alternative blocking group or 3′-O-alternative blocking group with a second label having a complementary moiety to form a polynucleotide probe having a plurality of labels; and (e) enzymatically cleaving the polynucleotide probe from the initiator.

In another aspect, the invention includes kits and compositions of matter for carrying out methods of the invention. In particular, the invention includes a composition of matter comprising 3′-O—NH₂-alkyne-nucleoside triphosphates for use in the enzymatic synthesis of polynucleotide probes, wherein the alkyne moiety is attached to a base via a linker. The invention further includes a kit for enzymatically synthesizing a polynucleotide probe comprising at least one 3′-O-blocked-(alkyne/azide)-nucleoside triphosphate or 3′-O-blocked-(protected alkyne/protected azide)-nucleoside triphosphate and a nucleoside triphosphate having a 3′-alternative blocking group or 3′-O-alternative blocking group, wherein the alternative blocking group has a reactive moiety and may be the same or different than a 3′-O-blocking group of the 3′-O-blocked-(alkyne/azide)-nucleoside triphosphate or 3′-O-blocked-(protected alkyne/protected azide)-nucleoside triphosphate.

The invention is in part based on the recognition and appreciation that template-free DNA polymerases, especially terminal deoxynucleotidyl transferase (TdT) variants, and endonuclease V enzymes can accommodate substrates that have been drastically altered (e.g., labels or “click” groups added) for the purpose of making modified natural compounds, such as polynucleotide probes, which have useful applications in a wide variety of fields, especially in the medical and health related fields.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the present invention.

### BRIEF DESCRIPTION OF THE DRAWINGS

FIG. 1A illustrates the basic steps of template-free enzymatic synthesis of polynucleotides.

FIGS. 1B-1D illustrate the steps of one embodiment of the method of the invention for synthesizing polynucleotide probes.

### DETAILED DESCRIPTION OF THE INVENTION

The general principles of the invention are disclosed in more detail herein particularly by way of examples, such as those shown in the drawings and described in detail. It should be understood, however, that the intention is not to limit the invention to the particular embodiments described. The invention is amenable to various modifications and alternative forms, specifics of which are shown for several embodiments. The intention is to cover all modifications, equivalents, and alternatives falling within the principles and scope of the invention.

The practice of the present invention may employ, unless otherwise indicated, conventional techniques and descriptions of organic chemistry, molecular biology (including recombinant techniques), cell biology, and biochemistry, which are within the skill of the art. Such conventional techniques may include, but are not limited to, preparation and use of synthetic peptides, synthetic polynucleotides, monoclonal antibodies, nucleic acid cloning, amplification, sequencing and analysis, and related techniques. Protocols for such conventional techniques can be found in product literature from manufacturers and in standard laboratory manuals, such as Genome Analysis: A Laboratory Manual Series (Vols. I-IV); PCR Primer: A Laboratory Manual; and Molecular Cloning: A Laboratory Manual (all from Cold Spring Harbor Laboratory Press); Lutz and Bornscheuer, Editors, Protein Engineering Handbook (Wiley-VCH, 2009); Hermanson, Bioconjugate Techniques, Second Edition (Academic Press, 2008); and like references.

The present invention is directed to methods, kits and compositions for enzymatic synthesis of labeled polynucleotide probes that may be used, for example, in “Taqman” assays. As used herein, the term “polynucleotide probe” refers to a polynucleotide that has a predetermined nucleotide sequence (typically the complement of a target polynucleotide) and that has one or more labels covalently attached. Such labels may include dyes, linkers, reactive groups, and the like, as described more fully below. In some embodiments, polynucleotide probes of the invention each have a plurality of labels attached. In particular, polynucleotide probes each may have two labels attached. In some embodiments, the two labels may be attached such that one label is at a 5′ end of the polynucleotide probe and the other label at a 3′ end of the polynucleotide probe. In some embodiments, one label is attached to a 3′ terminal nucleotide and the second label is attached to a 5′ terminal nucleotide. In a particular embodiment, such first label is a fluorescent dye and such second label is a fluorescent quencher that is capable of quenching the fluorescent emissions of the first label. As used herein, the term “labels” refers to any moiety that may be used directly or indirectly to detect the presence and/or quantity of a target nucleic acid to which the polynucleotide probe is capable of hybridizing. Typically, such detection is based on the appearance of a fluorescent signal and the intensity of a fluorescent signal, respectively. In some embodiments, labels are selected from the group consisting of fluorescent dyes and fluorescent quenchers, with the proviso that a polynucleotide probe comprises at least one fluorescent dye. Such fluorescent dyes and fluorescent quenchers are commercially available, e.g. Molecular Probes (ThermoFisher). In some embodiments, the length of the polynucleotide probe is selected so that when hybridized to a target nucleic acid the fluorescent dye and the fluorescent quencher are within a fluorescence resonant energy transfer (FRET) distance of one another so that no or minimal fluorescent emissions are emitted from the probe.

As mentioned above, methods of the invention for synthesizing a polynucleotide probe comprise the general steps of: (a) providing an initiator having a 3′-penultimate deoxyinosine and a 3′-terminal nucleotide having a free 3′-hydroxyl; (b) repeating elongation cycles comprising steps of: (i) contacting under elongation conditions the initiator or elongated fragments having free 3′-O-hydroxyls with a 3′-O-blocked nucleoside triphosphate and a template-independent DNA polymerase so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, until the polynucleotide of the polynucleotide probe is formed, wherein (A) at least one of the 3′-O-blocked nucleoside triphosphate is a 3′-O-blocked-(alkyne/azide)-nucleoside triphosphate or a 3′-O-blocked-(protected alkyne/protected azide)-nucleoside triphosphate, (B) in a final elongation cycle a nucleoside triphosphate having a 3′-alternative blocking group or 3′-O-alternative blocking group is contacted with said elongated fragments having free 3′-hydroxyls, (C) the alternative blocking group has a reactive moiety and may be the same or different than the blocking group, and (D) said step of deblocking is omitted; (c) reacting a first label having an (alkyne/azide) group with the at least one incorporated 3′-blocked-(alkyne/azide)-nucleoside triphosphate; (d) reacting the reactive moiety of the 3′-alternative blocking group or 3′-O-alternative blocking group with a second label having a complementary moiety to form a polynucleotide probe having a plurality of labels; and (e) enzymatically cleaving the polynucleotide probe from the initiator.

Regarding step (a), in some embodiments, the initiator is attached by its 5′ end to a solid support which facilitates the addition and removal of reagents in the synthesis process. In some embodiments, the 3′ terminal nucleotide is thymidine. In part the invention was a discovery that endonuclease V was unexpectedly able to cleave a polynucleotide probe from the initiator even when the first nucleotide of the polynucleotide probe contained a bulky label, such as a fluorescent dye. Likewise, the invention is in part a discovery that terminal deoxynucleotidyl transferases (TdTs) were available that had the capability of incorporating 3′-O-blocked-(alkyne/azide)-nucleoside triphosphates.

Regarding step (b), part (A), as used herein, the expression “-(alkyne/azide)-” refers to the “click” pair consisting of the alkyne group and the azido group and circumstances in which the groups may be attached to either of two moieties intended to be ligated or coupled. That is, for example, the expression indicates that if it is desired to ligate chemical moieties R1 and R2, that it may be accomplished by either carrying out a “click” reaction between R1—N3 and R2—C≡CH or carrying out a “click” reaction between R1—C≡CH and R2—N3. Likewise, the expression “-(protected alkyne/protected azide)-” denotes the same relationship, except that deprotection must be carried out prior to performance of a “click” reaction. As used herein, the step of reacting or implementing a “click” reaction in reference to alkyne and azide groups means a (3+2) cycloaddition reaction between azide and alkyne groups forming a 1,2,3-triazole ring, e.g. Sharpless et al, U.S. Pat. No. 7,763,736; Carell et al, U.S. Pat. No. 8,129,315; and the like, which are incorporated herein by reference.

In further regard to step (b), part (A), in some embodiments, 3′-O-blocked-(alkyne/azide)-nucleoside triphosphates are 3′-O—NH2-alkyne-nucleoside triphosphates. Such monomers may be synthesized from 3′-oxime-nucleoside starting materials using conventional chemistry, e.g. Winz et al, Nucleic Acids Research, 43(17): e110 (2015); Carell et al, U.S. Pat. No. 8,129,315; Sharpless et al, U.S. Pat. No. 7,375,234; and the like, which are incorporated herein by reference. For example, 3′—O—NH2-5-alkyne-deoxyuridine triphosphates (5-octa(1,7)diynyl-3′aminoxy-dUTP) may be synthesized from 3′-oxime-5-iodo-deoxyuridine starting material. Typically, alkyne groups and azide groups are attached to nucleobases, usually via a linker, at conventional positions for labeling nucleotides, e.g. 7-purine and 5-pyrimidine.

Regarding step (b), elongation cycles may also include one or more washing steps. In some embodiments, elongation cycles include a single washing step implemented after the deprotection step. In other embodiments, elongation cycles may include a washing step after each “contacting” step as well as after each “deblocking” step. In still further embodiments employing additional steps, such as capping steps, further washing steps may be implemented.

Regarding step (b), parts (B) and (C), in the final elongation cycle a monomer is added to which a second label is covalently coupled through an alternative blocking group. The alternative blocking group may be the same or different than the blocking group of the other monomers (i.e., non-final monomers). For example, when 3′-O—NH2-dNTPs or 3′-O-azidomethyl-dNTPs are used, then the “alternative blocking” group may be the same as the “blocking” groups because both the —NH2 and the -azidomethyl groups have a blocking function and are reactive to selected complementary groups. However, if the blocking groups are not reactive to a selected complementary group, then it is advantageous to employ an alternative blocking group in the monomer of the final cycle which has such reactivity, e.g. an azidomethyl or an amine. In some embodiments, the alternative blocking group of the final monomer may either be attached directly to the 3′ carbon nucleoside triphosphate (e.g. Lui et al, Nucleic Acids Resesarch, 35(21): 7140-7149 (2007) disclosing 3′-azido-(2′,3′)-dideoxy-dNTPs) or may be attached indirectly to the 3′ carbon via the 3′ oxygen (e.g. Guo et al, Acc. Chem. Res., 43(4): 551-563 (2010) disclosing 3′-O-azidomethyl-dNTPs). Thus, in some embodiments, the final monomer of the polynucleotide may be a 3′ -azido-2′,3′-dideoxynucleoside triphosphate. Or, alternatively, such monomer may be a 3′-O-azidomethyl-nucleoside triphosphate. Either case is advantageous since labels having an alkyne group may be readily coupled to the 3′ end of the polynucleotide probe via a “click” reaction.

Regarding step (d), whenever the 3′-oxygen of monomers are protected (or blocked) with amine groups, the blocking groups and alternative blocking groups may be the same and the complementary moiety of the second label may be an aldehyde group. In some embodiments, there is a single first label which is a fluorescent dye and a second label which is a fluorescent quencher capable of quenching the fluorescent emissions of the selected fluorescent dye. Among such embodiments, a fluorescent quencher may be a Black Hole Quencher (BHQ) dye. Azide derivatives of BHQ dyes may be synthesized as disclosed in Chevalier et al, Chem. Eur. J., 19: 1686-1699 (2013). The azide-BHQ dyes then may be converted into aldehyde-BHQ dyes by click reacting an alkyne-aldehyde linker (e.g. Biosearch Technologies) with the azide-BHQ dye. In other embodiments, the first label may be selected from the group of fluorescent dyes consisting of 6-carboxyfluoresein (FAM) and tetrachlorofluorescein (TET) and a second label may be tetramethylrhodamine (TAN/IRA).

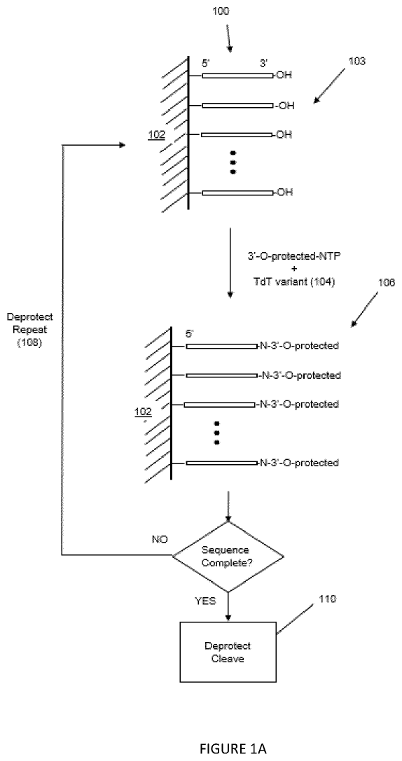

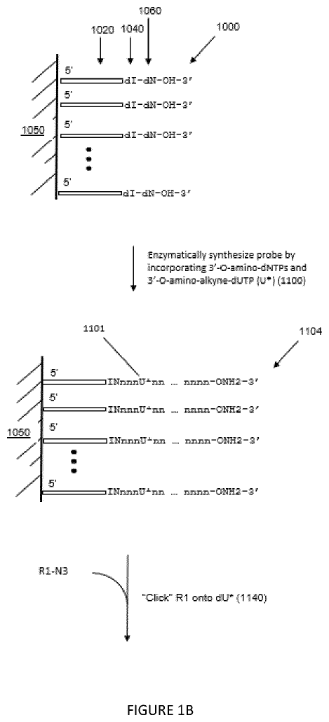

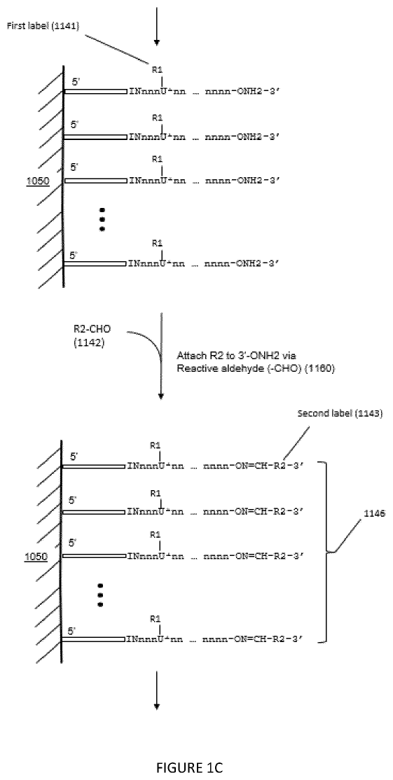

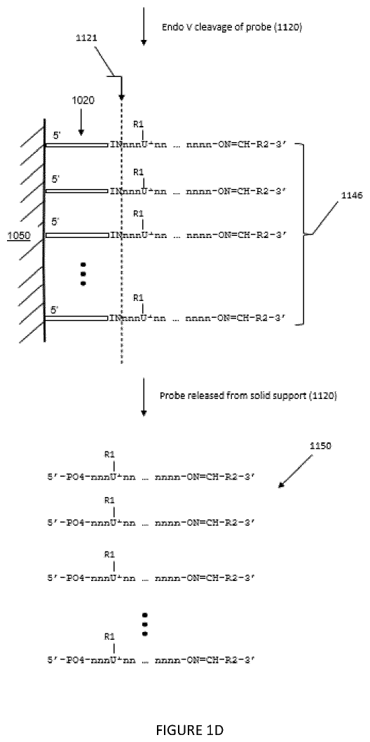

Regarding step (e), enzymatic cleavage may be effected by a variety of enzymes and corresponding cleavable nucleotides or linkages. In some embodiments, enzymatic cleavage is carried out with an endonuclease V activity that cleaves a single stranded DNA containing a deoxyinosine, as described in FIGS. 1B-1D. In other embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage by glycosylases and/or endonucleases may require a double stranded DNA substrate. In other embodiments, cleavable nucleotides include nucleotides comprising base analogs cleavable by endonuclease III which include, but are not limited to, urea, thymine glycol, methyl tartonyl urea, alloxan, uracil glycol, 6-hydroxy-5,6-dihydrocytosine, 5-hydroxyhydantoin, 5-hydroxycytocine, trans-1-carbamoyl-2-oxo-4,5-dihydrooxyimidazolidine, 5,6-dihydrouracil, 5 -hydroxycytosine, 5-hydroxyuracil, 5-hydroxy-6-hydrouracil, 5-hydroxy-6-hydrothymine, 5,6-dihydrothymine. In some embodiments, cleavable nucleotides include nucleotides comprising base analogs cleavable by formamidopyrimidine DNA glycosylase which include, but are not limited to, 7,8-dihydro-8-oxoguanine, 7,8-dihydro-8-oxoinosine, 7,8-dihydro-8-oxoadenine, 7,8-dihydro-8-oxonebularine, 4,6-diamino-5-formamidopyrimidine, 2,6-diamino-4-hydroxy-5-formamidopyrimidine, 2,6-diamino-4-hydroxy-5-N-methylformamidopyrimidine, 5-hydroxycytosine, 5-hydroxyuracil. In some embodiments, cleavable nucleotides include nucleotides comprising base analogs cleavable by hNeil 1 which include, but are not limited to, guanidinohydantoin, spiroiminodihydantoin, 5-hydroxyuracil, thymine glycol. In some embodiments, cleavable nucleotides include nucleotides comprising base analogs cleavable by thymine DNA glycosylase which include, but are not limited to, 5-formylcytosine and 5-carboxycytosine. In some embodiments, cleavable nucleotides include nucleotides comprising base analogs cleavable by human alkyladenine DNA glycosylase which include, but are not limited to, 3-methyladenine, 3-methylguanine, 7-methylguanine, 7-(2-chloroehyl)-guanine, 7-(2-hydroxyethyl)-guanine, 7-(2-ethoxyethyl)-guanine, 1,2-bis-(7-guanyl)ethane, 1,N6-ethenoadenine, 1,N2-ethenoguanine, N2,3-ethenoguanine, N2,3-ethanoguanine, 5-formyluracil, 5-hydroxymethyluracil, hypoxanthine. In some embodiments, cleavable nucleotides include 5-methylcytosine cleavable by 5-methylcytosine DNA glycosylase

FIGS. 1B-1D illustrate the steps of an exemplary embodiment of the method of template-free enzymatic synthesis of labeled polynucleotide probes which employs initiators with a penultimate deoxyinosine, 3′-O—NH2-nucleoside triphosphate monomers, alkyne-derivatized monomers, and an aldehyde-derivatized label to couple to the 3′-O-amine of the final nucleotide of the probe. FIG. 1B shows initiators (1000) attached by their 5′ ends to solid support (1050). Each initiator (1020) has a 3′-penultimate deoxyinosine (1040) next to 3′-terminal nucleotide (1060) that has a free 3′ hydroxyl. Such initiators on solid supports (which is a starting material for the present invention) may be produced using conventional chemical nucleic acid synthesis. After a predetermined number of cycles of enzymatic incorporation and de-blocking (1100), except for the final cycle that omits the de-blocking step, a polynucleotide product (1104) having terminal 3′-O—NH2 groups is formed. At least one of the nucleotides incorporated is a 3′-O—NH2-(5-alkyne)-deoxyuridine triphosphate (designated as “U*”, 1101). At this point, in this embodiment, a first label is attached (1140) by “click” reacting an azide-dervatized first label (R1—N3) to the alkyne of U* to give first label (1141). After first label (1141) is attached, aldehyde-derivative (1142) of second label (1143) is reacted (1160) with the 3′-O amines to form dual labeled probe (1146)

Dual labeled polynucleotide probe (1146) is then cleaved from initiators (1020) and support (1050) by treating the attached product with an endonuclease V activity which recognizes the presence of the deoxyinosine and cleaves the strand on the 3′ side (1121 in FIG. 1D) of terminal nucleotide (1060) of the initiators. In some embodiments, the endonuclease V activity is provided by using a prokaryotic endonuclease V. In still other embodiments, the endonuclease V is an E. coli endonuclease V. As used herein, the term “endonuclease V activity” means an enzyme activity that catalyzes the following cleavage reaction in a single stranded DNA: 5′ . . . NNINNNN . . . -3′→5′ - . . . NNIN+5′-PO₄-NNNN . . . -3′ where N is any nucleotide and I is deoxyinosine. Cleavage (1120) of polynucleotides (1146) by an endonuclease V activity releases polynucleotide probe (1150) which includes a 5′-monophosphate that optionally may be removed by a step of treating with a 5′-phosphatase.

Enzymes with endonuclease V activity are available from commercial enzyme suppliers, for example, New England Biolabs (Beverly, MA, USA), NzyTech (Lisbon, Portugal). Such enzymes may be used with the supplier's recommended cleavage buffers (e.g. 50 mM K—Ac, 20 mM Tris-Ac, 10 mM Mg—Ac, 1 mM DTT at pH 7.9). Typical cleavage conditions are as follows: 70U of Endo V in 500 of Nzytech buffer at 37° C. for 500 pmol synthesis scale on resin. Typical cleavage times are from 5 to 60 minutes, or from 10 to 30 minutes. Optionally, endonuclease activity of the above enzymes may be heat inactivated by incubation at 65° C. or higher for 20 minutes. Optionally, the Nzytech endonuclease V comprises a His tag that allows convenient removal of the enzyme from reaction mixtures in preparation of final products.

### Template-Free Enzymatic Synthesis

Generally, methods of template-free (or equivalently, “template-independent”) enzymatic DNA synthesis comprise repeated cycles of steps, such as are illustrated in FIG. 1A, in which a predetermined nucleotide is coupled to an initiator or growing chain in each cycle. The general elements of template-free enzymatic synthesis is described in the following references: Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57: 1821-1832 (2018); Mathews et al, Organic & Biomolecular Chemistry, DOI: 0.1039/c6ob01371f (2016); Schmitz et al, Organic Lett., 1(11): 1729-1731 (1999).

Initiator polynucleotides (100) are provided, for example, attached to solid support (102), which have free 3′-hydroxyl groups (103). To the initiator polynucleotides (100) (or elongated initiator polynucleotides in subsequent cycles) are added a 3′-O-protected-dNTP and a template-free polymerase, such as a TdT or variant thereof (e.g. Ybert et al, WO/2017/216472; Champion et al, WO2019/135007) under conditions (104) effective for the enzymatic incorporation of the 3′-O-protected-dNTP onto the 3′ end of the initiator polynucleotides (100) (or elongated initiator polynucleotides). This reaction produces elongated initiator polynucleotides whose 3′-hydroxyls are protected (106). If the elongated sequence is not complete, then another cycle of addition is implemented (108). If the elongated initiator polynucleotide contains a competed sequence, then the 3′-O-protection group may be removed, or deprotected, and the desired sequence may be cleaved from the original initiator polynucleotide (110). Such cleavage may be carried out using any of a variety of single strand cleavage techniques, for example, by inserting a cleavable nucleotide at a predetermined location within the original initiator polynucleotide. An exemplary cleavable nucleotide may be a uracil nucleotide which is cleaved by uracil DNA glycosylase. If the elongated initiator polynucleotide does not contain a completed sequence, then the 3′-O-protection groups are removed to expose free 3′-hydroxyls (103) and the elongated initiator polynucleotides are subjected to another cycle of nucleotide addition and deprotection.

As used herein, the terms “protected” and “blocked” in reference to specified groups, such as, a 3′-hydroxyls of a nucleotide or a nucleoside, are used interchangeably and are intended to mean a moiety is attached covalently to the specified group that prevents a chemical change to the group during a chemical or enzymatic process. Whenever the specified group is a 3′-hydroxyl of a nucleoside triphosphate, or an extended fragment (or “extension intermediate”) in which a 3′-protected (or blocked)-nucleoside triphosphate has been incorporated, the prevented chemical change is a further, or subsequent, extension of the extended fragment (or “extension intermediate”) by an enzymatic coupling reaction.

As used herein, an “initiator” (or equivalent terms, such as, “initiating fragment,” “initiator nucleic acid,” “initiator oligonucleotide,” or the like) usually refers to a short oligonucleotide sequence with a free 3′-hydroxyl at its end, which can be further elongated by a template-free polymerase, such as TdT. In one embodiment, the initiating fragment is a DNA initiating fragment. In an alternative embodiment, the initiating fragment is an RNA initiating fragment. In some embodiments, an initiating fragment possesses between 3 and 100 nucleotides, in particular between 3 and 20 nucleotides. In some embodiments, the initiating fragment is single-stranded. In alternative embodiments, the initiating fragment may be double-stranded. In some embodiments, an initiator oligonucleotide may be attached to a synthesis support by its 5′end; and in other embodiments, an initiator oligonucleotide may be attached indirectly to a synthesis support by forming a duplex with a complementary oligonucleotide that is directly attached to the synthesis support, e.g. through a covalent bond. In some embodiments a synthesis support is a solid support which may be a discrete region of a solid planar solid, or may be a bead.

In some embodiments, an initiator may comprise a non-nucleic acid compound having a free hydroxyl to which a TdT may couple a 3′-O-protected dNTP, e.g. Baiga, U.S. patent publications US2019/0078065 and US2019/0078126.

After synthesis is completed polynucleotides with the desired nucleotide sequence may be released from initiators and the solid supports by cleavage. A wide variety of cleavable linkages or cleavable nucleotides may be used for this purpose. In some embodiments, cleaving the desired polynucleotide leaves a natural free 5′-hydroxyl on a cleaved strand; however, in alternative embodiments, a cleaving step may leave a moiety, e.g. a 5′ -phosphate, that may be removed in a subsequent step, e.g. by phosphatase treatment.

Returning to FIG. 1A, in some embodiments, an ordered sequence of nucleotides are coupled to an initiator nucleic acid using a template-free polymerase, such as TdT, in the presence of 3′-O-protected dNTPs in each synthesis step. In some embodiments, the method of synthesizing an oligonucleotide comprises the steps of (a) providing an initiator having a free 3′-hydroxyl (100); (b) reacting (104) under extension conditions the initiator or an extension intermediate having a free 3′-hydroxyl with a template-free polymerase in the presence of a 3′-O-protected nucleoside triphosphate to produce a 3′-O-protected extension intermediate (106); (c) deprotecting the extension intermediate to produce an extension intermediate with a free 3′-hydroxyl (108); and (d) repeating steps (b) and (c) (110) until the polynucleotide is synthesized. (The terms “extension intermediate” and “elongation fragment” may be used interchangeably). In some embodiments, an initiator is provided as an oligonucleotide attached to a solid support, e.g. by its 5′ end. The above method may also include washing step after each reaction, or extension, step, as well as after each de-protecting step. For example, the step of reacting may include a sub-step of removing unincorporated nucleoside triphosphates, e.g. by washing, after a predetermined incubation period, or reaction time. Such predetermined incubation periods or reaction times may be a few seconds, e.g. 30 sec, to several minutes, e.g. 30 min.

When the sequence of polynucleotides on a synthesis support includes reverse complementary subsequences, secondary intra-molecular or cross-molecular structures may be created by the formation of hydrogen bonds between the reverse complementary regions. In some embodiments, base protecting moieties for exocyclic amines are selected so that hydrogens of the protected nitrogen cannot participate in hydrogen bonding, thereby preventing the formation of such secondary structures. That is, base protecting moieties may be employed to prevent the formation of hydrogen bonds, such as are formed in normal base pairing, for example, between nucleosides A and T and between G and C. At the end of a synthesis, the base protecting moieties may be removed and the polynucleotide product may be cleaved from the solid support, for example, by cleaving it from its initiator.

In addition to providing 3′-O-blocked dNTP monomers with base protection groups, elongation reactions may be performed at higher temperatures using thermal stable template-free polymerases. For example, a thermal stable template-free polymerase having activity above 40° C. may be employed; or, in some embodiments, a thermal stable template-free polymerase having activity in the range of from 40-85° C. may be employed; or, in some embodiments, a thermal stable template-free polymerase having activity in the range of from 40-65° C. may be employed.

In some embodiments, elongation conditions may include adding solvents to an elongation reaction mixture that inhibit hydrogen bonding or base stacking. Such solvents include water miscible solvents with low dielectric constants, such as dimethyl sulfoxide (DMSO), methanol, and the like. Likewise, in some embodiments, elongation conditions may include the provision of chaotropic agents that include, but are not limited to, n-butanol, ethanol, guanidinium chloride, lithium perchlorate, lithium acetate, magnesium chloride, phenol, 2-propanol, sodium dodecyl sulfate, thiourea, urea, and the like. In some embodiments, elongation conditions include the presence of a secondary-structure-suppressing amount of DMSO. In some embodiments, elongation conditions may include the provision of DNA binding proteins that inhibit the formation of secondary structures, wherein such proteins include, but are not limited to, single-stranded binding proteins, helicases, DNA glycolases, and the like.

3′-O-blocked dNTPs without base protection may be purchased from commercial vendors or synthesized using published techniques, e.g. U.S. Pat. No. 7,057,026; Guo et al, Proc. Natl. Acad. Sci., 105(27): 9145-9150 (2008); Benner, U.S. Pat. Nos. 7,544,794 and 8,212,020; International patent publications WO2004/005667, WO91/06678; Canard et al, Gene (cited herein); Metzker et al, Nucleic Acids Research, 22: 4259-4267 (1994); Meng et al, J. Org. Chem., 14: 3248-3252 (3006); U.S. patent publication 2005/037991. 3′-O-blocked dNTPs with base protection may be synthesized as described below.

When base-protected dNTPs are employed the above method of FIG. 1A may further include a step (e) removing base protecting moieties, which in the case of acyl or amidine protection groups may (for example) include treating with concentrated ammonia. The above method may also include one or more capping steps in addition to washing steps after the reacting, or extending, step A first capping step may cap, or render inert to further extensions, unreacted 3′-OH groups on partially synthesized polynucleotides. Such capping step is usually implemented after a coupling steps, and whenever a capping compound is used, it is selected to be unreactive with protection groups of the monomer just coupled to the growing strands. In some embodiments, such capping steps may be implemented by coupling (for example, by a second enzymatic coupling step) a capping compound that renders the partially synthesized polynucleotide incapable of further couplings, e.g. with TdT. Such capping compounds may be a dideoxynucleoside triphosphate. In other embodiments, non-extended strands with free 3′-hydroxyls may be degraded by treating them with a 3′ -exonuclease activity, e.g. Exo I. For example, see Hyman, U.S. Pat. No. 5,436,143. Likewise, in some embodiments, strands that fail to be deblocked may be treated to either remove the strand or render it inert to further extensions. A second capping step may be implemented after a deprotection step, to render the affected strands inert from any subsequent coupling or deprotection any 3′-O protection, or blocking groups. Capping compounds of such second capping step are selected so that they do not react with free 3′-hydroxyls that may be present. In some embodiments, such second capping compound may be a conjugate of an aldehyde group and a hydrophobic group. The latter group permits separation based on hydrophobicity, e.g. Andrus, U.S. Pat. No. 5,047,524.

In some embodiments, reaction conditions for an elongation step (also sometimes referred to as an extension step or a coupling step) may comprising the following: 2.0 μM purified TdT; 125-600 μM 3′-O-blocked dNTP (e.g. 3′-O—NH₂-blocked dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out in a 50 μL reaction volume, at a temperature within the range RT to 45° C., for 3 minutes. In embodiments, in which the 3′-O-blocked dNTPs are 3′-O—NH₂-blocked dNTPs, reaction conditions for a deblocking step may comprise the following: 700 mM NaNO₂; 1 M sodium acetate (adjusted with acetic acid to pH in the range of 4.8-6.5), where the deblocking reaction may be carried out in a 50 μL volume, at a temperature within the range of RT to 45° C. for 30 seconds to several minutes. Washes may be performed with the cacodylate buffer without the components of the coupling reaction (e.g. enzyme, monomer, divalent cations).

Depending on particular applications, the steps of deblocking and/or cleaving may include a variety of chemical or physical conditions, e.g. light, heat, pH, presence of specific reagents, such as enzymes, which are able to cleave a specified chemical bond. Guidance in selecting 3′-O-blocking groups and corresponding de-blocking conditions may be found in the following references, which are incorporated by reference: Benner, U.S. Pat. Nos. 7,544,794 and 8,212,020; 5,808,045; 8,808,988; International patent publication WO91/06678; and references cited below. In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl)phosphine (TCEP) can be used to cleave a 3′O-azidomethyl groups, palladium complexes can be used to cleave a 3′O-allyl groups, or sodium nitrite can be used to cleave a 3′O-amino group. In particular embodiments, the cleaving reaction involves TCEP, a palladium complex or sodium nitrite.

As noted above, in some embodiments it is desirable to employ two or more blocking groups that may be removed using orthogonal de-blocking conditions. The following exemplary pairs of blocking groups may be used in parallel synthesis embodiments. It is understood that other blocking group pairs, or groups containing more than two, may be available for use in these embodiments of the invention.

| | |
|---|---|
| 3′-O-NH2 | 3′-O-azidomethyl |
| 3′-O-NH2 | 3′-O-allyl, 3′-O-propargyl |
| 3′-O-NH2 | 3′-O-phosphate |
| 3′-O-azidomethyl | 3′-O-allyl, 3′O-propargyl |
| 3′-O-azidomethyl | 3′-O-phosphate |
| 3′-O-allyl, 3′O-propargyl | 3′-O-phosphate |

Synthesizing oligonucleotides on living cells requires mild deblocking, or deprotection, conditions, that is, conditions that do not disrupt cellular membranes, denature proteins, interfere with key cellular functions, or the like. In some embodiments, deprotection conditions are within a range of physiological conditions compatible with cell survival. In such embodiments, enzymatic deprotection is desirable because it may be carried out under physiological conditions. In some embodiments specific enzymatically removable blocking groups are associated with specific enzymes for their removal. For example, ester- or acyl-based blocking groups may be removed with an esterase, such as acetylesterase, or like enzyme, and a phosphate blocking group may be removed with a 3′ phosphatase, such as T4 polynucleotide kinase. By way of example, 3′-O-phosphates may be removed by treatment with as solution of 100 mM Tris-HCl (pH 6.5) 10 mM MgCl₂, 5 mM 2-mercaptoethanol, and one Unit T4 polynucleotide kinase. The reaction proceeds for one minute at a temperature of 37° C.

A “3′-phosphate-blocked” or “3′-phosphate-protected” nucleotide refers to nucleotides in which the hydroxyl group at the 3′-position is blocked by the presence of a phosphate containing moiety. Examples of 3′-phosphate-blocked nucleotides in accordance with the invention are nucleotidyl-3′-phosphate monoester/nucleotidyl-2′,3′-cyclic phosphate, nucicotidyl-2′-phosphate monoester and nucleotidyl-2′ or 3′-alkylphosphate diester, and nucleotidyl-2′ or 3′-pyrophosphate. Thiophosphate or other analogs of such compounds can also be used, provided that the substitution does not prevent dephosphorylation resulting in a free 3′-OH by a phosphatase.

Further examples of synthesis and enzymatic deprotection of 3′-O-ester-protected dNTPs or 3′-O-phosphate-protected dNTPs are described in the following references: Canard et al, Proc. Natl. Acad. Sci., 92:10859-10863 (1995); Canard et al, Gene, 148: 1-6 (1994); Cameron et al, Biochemistry, 16(23): 5120-5126 (1977); Rasolonjatovo et al, Nucleosides & Nucleotides, 18(4&5): 1021-1022 (1999); Ferrero et al, Monatshefte fur Chemie, 131: 585-616 (2000); Taunton-Rigby et al, J. Org. Chem., 38(5): 977-985 (1973); Uemura et al, Tetrahedron Lett., 30(29): 3819-3820 (1989); Becker et al, J. Biol. Chem., 242(5): 936-950 (1967); Tsien, International patent publication W01991/006678.

In some embodiments, the modified nucleotides comprise a modified nucleotide or nucleoside molecule comprising a purine or pyrimidine base and a ribose or deoxyribose sugar moiety having a removable 3′-OH blocking group covalently attached thereto, such that the 3′ carbon atom has attached a group of the structure:

-O-Z

wherein -Z is any of —C(R′)₂—O—R″, —C(R′)₂—N(R″)₂, —C(R′)₂—N(H)R″, —C(R′)₂—S—R″ and —C(R′)₂—F, wherein each R″ is or is part of a removable protecting group; each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; with the proviso that in some embodiments such substituents have up to 10 carbon atoms and/or up to 5 oxygen or nitrogen heteroatoms; or (R′)₂ represents a group of formula ═C(R′″)₂ wherein each R′″ may be the same or different and is selected from the group comprising hydrogen and halogen atoms and alkyl groups, with the proviso that in some embodiments the alkyl of each R″ has from 1 to 3 carbon atoms; and wherein the molecule may be reacted to yield an intermediate in which each R″ is exchanged for H or, where Z is —(R′)₂—F, the F is exchanged for OH, SH or NH₂, preferably OH, which intermediate dissociates under aqueous conditions to afford a molecule with a free 3′-OH; with the proviso that where Z is —C(R′)₂—S—R″, both R′ groups are not H. In certain embodiments, R′ of the modified nucleotide or nucleoside is an alkyl or substituted alkyl, with the proviso that such alkyl or substituted alkyl has from 1 to 10 carbon atoms and from 0 to 4 oxygen or nitrogen heteroatoms. In certain embodiments, -Z of the modified nucleotide or nucleoside is of formula —C(R′)₂—N₃. In certain embodiments, Z is an azidomethyl group.

In some embodiments, Z is a cleavable organic moiety with or without heteroatoms having a molecular weight of 200 or less. In other embodiments, Z is a cleavable organic moiety with or without heteroatoms having a molecular weight of 100 or less. In other embodiments, Z is a cleavable organic moiety with or without heteroatoms having a molecular weight of 50 or less. In some embodiments, Z is an enzymatically cleavable organic moiety with or without heteroatoms having a molecular weight of 200 or less. In other embodiments, Z is an enzymatically cleavable organic moiety with or without heteroatoms having a molecular weight of 100 or less. In other embodiments, Z is an enzymatically cleavable organic moiety with or without heteroatoms having a molecular weight of 50 or less. In other embodiments, Z is an enzymatically cleavable ester group having a molecular weight of 200 or less. In other embodiments, Z is a phosphate group removable by a 3′-phosphatase. In some embodiments, one or more of the following 3′-phosphatases may be used with the manufacturer's recommended protocols: T4 polynucleotide kinase, calf intestinal alkaline phosphatase, recombinant shrimp alkaline phosphatase (e.g. available from New England Biolabs, Beverly, MA).

In a further embodiment, the 3′-blocked nucleotide triphosphate is blocked by either a 3′-O-azidomethyl, 3′-O—NH₂ or 3′-O-allyl group. In other embodiments, 3′-O-blocking groups of the invention include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), 3′-O-nitro, and 3′-O-propargyl. In other embodiments, the 3′-blocked nucleotide triphosphate is blocked by either a 3′-O-azidomethyl or a 3′-O—NH₂. Synthesis and use of such 3′-blocked nucleoside triphosphates are disclosed in the following references: U.S. Pat. Nos. 9,410,197; 8,808,988; 6,664,097; 5,744,595; 7,544,794; 8,034,923; 8,212,020; 10,472,383; Guo et al, Proc. Natl. Acad. Sci., 105(27): 9145-9150 (2008); and like references.

In some embodiments, 3′-O— protection groups are electrochemically labile groups. That is, deprotection or cleavage of the protection group is accomplished by changing the electrochemical conditions in the vicinity of the protection group which result in cleavage. Such changes in electrochemical conditions may be brought about by changing or applying a physical quantity, such as a voltage difference or light to activate auxiliary species which, in turn, cause changes in the electrochemical conditions at the site of the protection group, such as an increase or decrease in pH. In some embodiments, electrochemically labile groups include, for example, pH-sensitive protection groups that are cleaved whenever the pH is changed to a predetermined value. In other embodiments, electrochemically labile groups include protecting groups which are cleaved directly whenever reducing or oxidizing conditions are changed, for example, by increasing or decreasing a voltage difference at the site of the protection group.

In some embodiments, enzymatic synthesis methods employ TdT variants that display increased incorporation activity with respect to 3′-O-modified nucleoside triphosphates. For example, such TdT variants may be produced using techniques described in Champion et al, U.S. Pat. No. 10,435,676, which is incorporated herein by reference. In some embodiments, a TdT variant is employed having an amino acid sequence at least 60 percent identical to SEQ ID NO: 2 and a substitution at a first arginine at position 207 and a substitution at a second arginine at position 325, or functionally equivalent residues thereof. In some embodiments, a terminal deoxynucleotidyl transferase (TdT) variant is employed that has an amino acid sequence at least sixty percent identical to an amino acid sequence selected from SEQ ID NO: 2, 3, 4, 5, 6, 7, 8, 9, 10, 11, 12, 13, 14 or 15 with a substitution of arginine (“first arginine”) at position 207 with respect to SEQ ID NOs 2, 3, 4, 6, 7, 9, 12 and 13, at position 206 with respect to SEQ ID NO 5, at position 208 with respect to SEQ ID NOs 8 and 10, at position 205 with respect to SEQ ID NO 11, at position 216 with respect to SEQ ID NO 14 and at position 210 with respect to SEQ ID NO 15; and a substitution of arginine (“second arginine”) at position 325 with respect to SEQ ID NOs 2, 9 and 13, at position 324 with respect to SEQ ID NOs 3 and 4, at position 320 with respect to SEQ ID NO 320, at position 331 with respect to SEQ ID NOs 6 and 8, at position 323 with respect to SEQ ID NO 11, at position 328 with respect to SEQ ID NOs 12 and 15, and at position 338 with respect to SEQ ID NO 14; or functionally equivalent residues thereof; wherein the TdT variant (i) is capable of synthesizing a nucleic acid fragment without a template and (ii) is capable of incorporating a 3′-O-modified nucleotide onto a free 3′-hydroxyl of a nucleic acid fragment. In some embodiments, the above percent identity value is at least 80 percent identity with the indicated SEQ ID NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As used herein, the percent identity values used to compare a reference sequence to a variant sequence do not include the expressly specified amino acid positions containing substitutions of the variant sequence; that is, the percent identity relationship is between sequences of a reference protein and sequences of a variant protein outside of the expressly specified positions containing substitutions in the variant. Thus, for example, if the reference sequence and the variant sequence each comprised 100 amino acids and the variant sequence had mutations at positions 25 and 81, then the percent homology would be in regard to sequences 1-24, 26-80 and 82-100.

In regard to (ii), such 3′-O-modified nucleotide may comprise a 3′-O—NH₂-nucleoside triphosphate, a 3′-O-azidomethyl-nucleoside triphosphate, a 3′-O-allyl-nucleoside triphosphate, a 3′O-(2-nitrobenzyl)-nucleoside triphosphate, or a 3′-O-propargyl-nucleoside triphosphate.

In some embodiments, the above TdT variants have substitutions at the first and second arginines as shown in Table 1.

TABLE 1

| SEQ | | | | | |
|---|---|---|---|---|---|
| ID | | | | | |
| NO | Substitutions | | | | |
| 1 | M192R/Q | C302G/R | R336L/N | R454P/N/A/V | E457N/L/T/S/K |
| 2 | M63R/Q | C173G/R | R207L/N | R325P/N/A/V | E328N/L/T/S/K |
| 3 | M63R/Q | C173G/R | R207L/N | R324P/N/A/V | E327N/L/T/S/K |
| 4 | M63R/Q | C173G/R | R207L/N | R324P/N/A/V | E327N/L/T/S/K |
| 5 | — | C172G/R | R206L/N | R320P/N/A/V | — |
| 6 | M63R/Q | C173G/R | R207L/N | R331P/N/A/V | E334N/L/T/S/K |
| 7 | M63R/Q | C173G/R | R207L/N | — | E328N/L/T/S/K |
| 8 | — | C174G/R | R208L/N | R331P/N/A/V | E334N/L/T/S/K |
| 9 | M73R/Q | C173G/R | R207L/N | R325P/N/A/V | E328N/L/T/S/K |
| 10 | M64R/Q | C174G/R | R208L/N | — | E329N/L/T/S/K |
| 11 | M61R/Q | C171G/R | R205L/N | R323P/N/A/V | E326N/L/T/S/K |
| 12 | M63R/Q | C173G/R | R207L/N | R328P/N/A/V | E331N/L/T/S/K |
| 13 | — | C173G/R | R207L/N | R325P/N/A/V | E328N/L/T/S/K |
| 14 | M63R/Q | C182G/R | R216L/N | R338P/N/A/V | E341N/L/T/S/K |
| 15 | M66R/Q | C176G/R | R210L/N | R328P/N/A/V | E331N/L/T/S/K |

In some embodiments, further TdT variants for use with methods of the invention include one or more of the further substitutions of methionine, cysteine or glutamic acid, as shown in Table 1.

Further specific TdT variants that may be used in methods of the invention are set forth in Table 2. Each of the TdT variants DS1001 through DS1018 of Table 2 comprises an amino acid sequence at least 60 percent identical to SEQ ID NO 2 and comprises the substitutions at the indicated positions. In some embodiments, TdT variants DS1001 through DS1018 comprises an amino acid sequence at least 80 percent identical to SEQ ID NO 2 and comprises the substitutions at the indicated positions; in some embodiments, TdT variants DS1001 through DS1018 comprises an amino acid sequence at least 90 percent identical to SEQ ID NO 2 and comprises the substitutions at the indicated positions; in some embodiments, TdT variants DS1001 through DS1018 comprises an amino acid sequence at least 95 percent identical to SEQ ID NO 2 and comprises the substitutions at the indicated positions; in some embodiments, TdT variants DS1001 through DS1018 comprises an amino acid sequence at least 97 percent identical to SEQ ID NO 2 and comprises the substitutions at the indicated positions; in some embodiments, TdT variants DS1001 through DS1018 comprises an amino acid sequence at least 98 percent identical to SEQ ID NO 2 and comprises the substitutions at the indicated positions; in some embodiments, TdT variants DS1001 through DS1018 comprises an amino acid sequence at least 99 percent identical to SEQ ID NO 2 and comprises the substitutions at the indicated positions.

TABLE 2
Specific TdT Variants for Use with Methods of the Invention

| | |
|---|---|
| DS1001 | A17V + L52F + M63R + A108V + C173G + R207L + K265T + G284P + |
| (TH M27) | E289V + R325P + E328N + R351K |
| DS1002 | A17V + Q37E + D41R + L52F + G57E + M63R + S94R + G98E + A108V + |
| (M44) | S119A + L131R + S146E + Q149R + C173G + R207L + K265T + G284P + |
| | E289V + R325P + Q326F + E328N + H337D + R351K + W377R |
| DS1003 | A17V + Q37E + D41R + L52F + G57E + M63R + S94R + G98E + A108V + |
| | S146E + Q149R + C173G + F193Y + V199M + M201V + R207L + K265T + |
| | G284P + E289V + Q326F + E328N + R351K |
| DS1004 | A17V + Q37E + D41R + L52F + G57E + M63R + S94R + G98E + A108V + |
| (M45) | S146E + Q149R + C173G + F193Y + V199M + M201V + R207L + K265T + |
| | G284P + E289V + R325A + Q326F + E328N + R351K |
| DS1005 | A17V + Q37E + D41R + L52F + G57E + M63R + S94R + G98E + A108V + |
| | S146E + Q149R + C173G + F193Y + V199M + M201V + R207L + K265T + |
| | G284P + E289V + Q326F + E328N + R351K |
| DS1006 | L52F + A108V + R351K + A17V + Q37E + D41R + G57E+ C59R + L60D + |
| (M46) | M63R + S94R + G98E + S119A + L131R + S146E + Q149R + C173G + |
| | R207L + K265T + G284P + E289V + R325A + Q326F + E328N |
| DS1007 | L52F + A108V + R351K + A17V + Q37E + D41R + G57E + C59R + L60D + |
| (M47) | M63R + S94R + G98E + K118Q + S119A + L131R + S146E + Q149R + |
| | C173G + R207L + K265T + G284P + E289V + R325A + Q326F + E328N + |
| | W377R |
| DS1008 | A17V + Q37E + D41R + L52F + G57E + C59R + L60D + M63R + S94R + |
| | G98E + A108V + S119A + L131R + S146E + Q149R + C173G + R207L + |
| | F259S + Q261L + G284P + E289V + R325A + Q326F + E328N + R351K + |
| | W377R |
| DS1009 | A17V + D41R + L53F + G57E + C59R + L60D + M63R + S94R + G98E + |
| (MS 13-34) | K118Q + S119A + L131R + S146E + Q149R + C173G + R207L + K265T + |
| | G284P + E289V + R325A + Q326F + R351K + W377R |
| DS1010 | A17V + D41R + L52F + G57E + C59R + L60D + M63R + S94R + G98E + |
| (MS 34-1) | A108V + S119A + L131R + S146E + Q149R + R207L + K265T + G284P + |
| | E289V + R325A + Q326F + R351K |
| DS1011 | A17V + D41R + L53F + G57E + C59R + L60D + M63R + S94R + G98E + |
| | K118Q + S119A + L131R + S146E + Q149R + C173G + R207L + K265T + |
| | G284P + E289V + Q326F + R351K + W377R |
| DS1012 | A17V + Q37E + D41R + L52F + G57E + C59R + L60D + M63R + S94R + |
| (M48) | G98E + A108V + S119A + L131R + S146E + Q149R + C173G + R207L + |
| | F259S + Q261L, G284P + E289V + R325A + Q326F + E328N + R351K + |
| | W377R |
| DS1013 | A17V + Q37E + D41R + L52F + G57E + M63R + S94R + G98E + A108V + |
| | S146E + Q149R + C173G + R207L + K265T + G284P + E289V + R325A + |
| | Q326F + E328N + R351K |
| DS1014 | A17V + Q37E + D41R + L52F + G57E + C59R + L60D + M63R + S94R + |
| (M49) | G98E + A108V + S119A + L131R + S146E + Q149R + C173G + R207L + |
| | E257D + F259S + K260R + Q261L + G284P + E289V + R325A + Q326F + |
| | E328N + R351K + W377R |
| DS1015 | A17V + Q37E + D41R + L52F + G57E + C59R + L60D + M63R + S94R + |
| | G98E + A108V + S119A + L131R + S146E + Q149R + C173G + F193Y + |
| | V199M + M201V + R207L + E257D + F259S + K260R + Q261L + G284P + |
| | E289V + R325A + Q326F + E328N + R351K + W377R |
| DS1016 | A17V + D41R + L52F + G57E + M63R + S94R + G98E + A108V + S146E + |
| TH c2_5 | Q149R + C173G + M184T + R207L + K209H + G284L + E289A + R325V + |
| | E328K + R351K |
| DS1017 | A17V + L52F + G57E + M63R + A108V + C173G + R207L + K265T + |
| (M27) | G284P + E289V + R325P + E328N + R351K |
| DS1018 | A17V + L32T + Q37R + D41R + L52F + G57E + C59R + L60D + M63R + |
| (M60) | S67A + S94R + G98E + A108V + S119A +L131R + S146E + Q149R + |
| | V171A + S172E + C173R + V182I + S183E + R207L + K209H + M210K + |
| | T211I + E223G + A224P + E228D + Q261L + G284P + E289V + R325A + |
| | Q326F + E328N + R351K + D372E |

TdT variants of the invention as described above each comprise an amino acid sequence having a percent sequence identity with a specified SEQ ID NO, subject to the presence of indicated substitutions. In some embodiments, the number and type of sequence differences between a TdT variant of the invention described in this manner and the specified SEQ ID NO may be due to substitutions, deletion and/or insertions, and the amino acids substituted, deleted and/or inserted may comprise any amino acid. In some embodiments, such deletions, substitutions and/or insertions comprise only naturally occurring amino acids. In some embodiments, substitutions comprise only conservative, or synonymous, amino acid changes, as described in Grantham, Science, 185: 862-864 (1974). That is, a substitution of an amino acid can occur only among members of its set of synonymous amino acids. In some embodiments, sets of synonymous amino acids that may be employed are set forth in Table 3A.

TABLE 3A
Synonymous Sets of Amino Acids I

| Amino Acid | Synonymous Set |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Gly, Ala, Thr, Pro, Ser |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Cys, Ser, Thr |
| His | His, Glu, Lys, Gln, Thr, Arg |
| Gln | Gln, Glu, Lys, Asn, His, Thr, Arg |
| Asn | Asn, Gln, Asp, Ser |
| Lys | Lys, Glu, Gln, His, Arg |
| Asp | Asp, Glu, Asn |
| Glu | Glu, Asp, Lys, Asn, Gln, His, Arg |
| Met | Met, Phe, Ile, Val, Leu |
| Trp | Trp |

In some embodiments, sets of synonymous amino acids that may be employed are set forth in Table 3B.

TABLE 3B
Synonymous Sets of Amino Acids II

| Amino Acid | Synonymous Set |
|---|---|
| Ser | Ser |
| Arg | Arg, Lys, His |
| Leu | Ile, Phe, Met, Leu |
| Pro | Ala, Pro |
| Thr | Thr |
| Ala | Pro, Ala |
| Val | Met, Ile Val |
| Gly | Gly |
| Ile | Met, Phe, Val, Leu, Ile |
| Phe | Met, Tyr, Ile, Leu, Phe |
| Tyr | Trp, Met |
| Cys | Cys, Ser |
| His | His, Gln, Arg |
| Gln | Gln, Glu, His |
| Asn | Asn, Asp |
| Lys | Lys, Arg |
| Asp | Asp, Asn |
| Glu | Glu, Gln |
| Met | Met, Phe, Ile, Val, Leu |
| Trp | Trp |

### Kits

The invention includes kits for carrying out methods of the invention. In one aspect, a kit for synthesizing a polynucleotide probe comprises at least one 3-O—NH2-alkyne-nucleoside triphosphate. In a particular embodiment, such 3-O—NH2-alkyne-nucleoside triphosphate includes a 5-octa(1,7)diynyl-3′aminoxy-dUTP.

In another aspect, a kit for enzymatically synthesizing a polynucleotide probe comprising at least one 3′-O-blocked-(alkyne/azide)-nucleoside triphosphate or 3′-O-blocked-(protected alkyne/protected azide)-nucleoside triphosphate and a nucleoside triphosphate having a 3′-alternative blocking group or 3′-O-alternative blocking group, wherein the alternative blocking group has a reactive moiety and may be the same or different than a 3′-O-blocking group of the 3′-O-blocked-(alkyne/azide)-nucleoside triphosphate or 3′-O-blocked-(protected alkyne/protected azide)-nucleoside triphosphate. In some embodiments, such at least one 3′-O-blocked-(alkyne/azide)-nucleoside triphosphate or 3′-O-blocked-(protected alkyne/protected azide)-nucleoside triphosphate is at least one 3-O—NH2-alkyne-nucleoside triphosphate, and such nucleoside triphosphate having a 3′-alternative blocking group or 3′-O-alternative blocking group is a 3′-O—NH2-nucleoside triphosphate. In some embodiments, such kit further comprises a second label with an aldehyde group capable of reacting with the 3′-O-amine of the polynucleotide probe. In some embodiments, such kit further includes an azide-derivatized first label capable of click reacting with the alkyne group incorporated into the polynucleotide probe.

In some embodiments, the above kits of the invention may further comprise an initiator attached to a support by a 5′ end and having a deoxyinosine penultimate to a 3′ end and free 3′-hydroxyl. In some embodiments, a kit of the invention further includes an endonuclease V capable of cleaving an initiator-polynucleotide conjugate 3′ of a terminal nucleotide of the initiator. In some such kits, the endonuclease V has a capture moiety to permit removal from a reaction mixture. In some kits, such capture moiety is a His tag. In some embodiments, initiators of a kit have a 3′-terminal sequence of 5′-dI-dT-3′. In some embodiments, initiators of a kit have a 3′-terminal sequence of 5′ -dI-dG-3′. In some embodiments, initiators of a kit have a 3′-terminal sequence of 5′-dI-dA-3′. In some embodiments, initiators of a kit have a 3′-terminal sequence of 5′-dI-dT-3′, 5′ -dI-dG-3′, or 5′-dI-dA-3′. In some embodiments, such support is a solid support. Such solid support may comprise beads, such as magnetic bead, a planar solid, such as a glass slide, or a membrane, or the like. In some embodiments, a kit of the invention may further include a template-free polymerase and 3′-O-blocked nucleoside triphosphates of one or more of deoxyadenosine, deoxyguanosine, thymidine, deoxyuridine and deoxycytidine. In some kits, such template-free polymerase may be a TdT. In some embodiments, such TdT may be a TdT variant described herein. In some embodiments, a kit of the invention may further include a de-blocking agent which is capable of removing 3′ blocking groups from incorporated 3′-O-blocked nucleotides.

Real Time PCR with Taqman Probes

Polynucleotide probes of the invention may be used in Taqman real-time PCR assays, for example, using commercially available kits. In some embodiments, a Taqman real-time PCR assay is the same as a PCR assay with the addition of a concentration of polynucleotide probe which is within about a factor of two of the primer concentrations. Preferably the amplified fragment is in the range of from about 50 to about 150 base pairs. The length of the polynucleotide probe may be in the range of from 10 to 30 nucleotides, or in the range of from 15 to 25 nucleotides, or in the range of from 20 to 22 nucleotides. Preferably the assay uses Taq DNA polymerase, or an equivalent polymerase, that has 5′->3′ exonuclease activity.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999).

“Functionally equivalent” in reference to amino acid positions in two or more different TdTs means (i) the amino acids at the respective positions play the same functional role in an activity of the TdTs, and (ii) the amino acids occur at homologous amino acid positions in the amino acid sequences of the respective TdTs. It is possible to identify positionally equivalent or homologous amino acid residues in the amino acid sequences of two or more different TdTs on the basis of sequence alignment and/or molecular modelling. In some embodiments, functionally equivalent amino acid positions belong to sequence motifs that are conserved among the amino acid sequences of TdTs of evolutionarily related species, e.g. genus, families, or the like. Examples of such conserved sequence motifs are described in Motea et al, Biochim. Biophys. Acta. 1804(5): 1151-1166 (2010); Delarue et al, EMBO J., 21: 427-439 (2002); and like references.

“Kit” refers to any delivery system for delivering materials or reagents for carrying out a method of the invention. In the context of reaction assays, such delivery systems include systems and/or compounds (such as dilutants, surfactants, carriers, or the like) that allow for the storage, transport, or delivery of reaction reagents (e.g., fluorescent labels, such as mutually quenching fluorescent labels, fluorescent label linking agents, enzymes, quenching agents, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second or more containers contain mutually quenching fluorescent labels and/or quenching agents.

“Mutant” or “variant,” which are used interchangeably, refer to polypeptides derived from a natural or reference TdT polypeptide described herein, and comprising a modification or an alteration, i.e., a substitution, insertion, and/or deletion, at one or more positions. Variants may be obtained by various techniques well known in the art. In particular, examples of techniques for altering the DNA sequence encoding the wild-type protein, include, but are not limited to, site-directed mutagenesis, random mutagenesis, sequence shuffling and synthetic oligonucleotide construction. Mutagenesis activities consist in deleting, inserting or substituting one or several amino-acids in the sequence of a protein or in the case of the invention of a polymerase. The following terminology is used to designate a substitution: L238A denotes that amino acid residue (Leucine, L) at position 238 of a reference, or wild type, sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference.

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Sequence identity” refers to the number (or fraction, usually expressed as a percentage) of matches (e.g., identical amino acid residues) between two sequences, such as two polypeptide sequences or two polynucleotide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or ttp://www.ebi.ac.uk/Tools/emboss/. Those skilled in the art can determine appropriate parameters for measuring alignment, including any algorithm needed to achieve maximal alignment over the full length of the sequences being compared. For purposes herein, % amino acid sequence identity values refer to values generated using the pair wise sequence alignment program EMBOSS Needle, that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

A “substitution” means that an amino acid residue is replaced by another amino acid residue. Preferably, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues. The sign “+” indicates a combination of substitutions. The amino acids are herein represented by their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr). In the present document, the following terminology is used to designate a substitution: L238A denotes that amino acid residue (Leucine, L) at position 238 of the parent sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

1-18. (canceled)

19. A method of synthesizing a polynucleotide probe having a predetermined sequence and a plurality of labels, the method comprising the steps of:
a) providing an initiator having a 3′-penultimate deoxyinosine and a 3′-terminal nucleotide having a free 3′ -hydroxyl;
b) repeating elongation cycles comprising steps of: (i) contacting under elongation conditions the initiator or elongated fragments having free 3′-O-hydroxyls with a 3′-O-blocked nucleoside triphosphate and a template-independent DNA polymerase so that the initiator or elongated fragments are elongated by incorporation of a 3′-O-blocked nucleoside triphosphate to form 3′-O-blocked elongated fragments, and (ii) deblocking the elongated fragments to form elongated fragments having free 3′ -hydroxyls, until the polynucleotide probe is formed, wherein (A) at least one of the 3′-O-blocked nucleoside triphosphate is a 3′-O-blocked-(alkyne/azide)-nucleoside triphosphate or a 3′-O-blocked-(protected alkyne/protected azide)-nucleoside triphosphate , (B) in a final elongation cycle a nucleoside triphosphate having a 3′ -alternative blocking group or 3′-O-alternative blocking group is contacted with said elongated fragments having free 3′-hydroxyls, (C) the alternative blocking group has a reactive moiety and may be the same or different than the blocking group, and (D) said step of deblocking is omitted;
c) reacting a first label having an (alkyne/azide) group with the at least one incorporated 3′ -blocked-(alkyne/azide)-nucleoside triphosphate;
d) reacting the reactive moiety of the 3′-alternative blocking group or 3′-O-alternative blocking group with a second label having a complementary moiety to form a polynucleotide probe having a plurality of labels; and
e) enzymatically cleaving the polynucleotide probe from the initiator.

20. The method of claim 19, wherein said step of enzymatically cleaving comprises treating said polynucleotide probe with an endonuclease V activity.

21. The method of claim 20, wherein (i) said endonuclease V activity is provided by a prokaryotic endonuclease V, (ii) said template-independent DNA polymerase is a terminal deoxynucleotidyl transferase (TdT), and (iii) said initiator is attached to a solid support by a 5′ end.

22. The method of claim 21, wherein said prokaryotic endonuclease V is an E. coli endonuclease V.

23. The method of claim 21, further including a step of removing said prokaryotic endonuclease V from said cleaved polynucleotide probe.

24. The method of claim 21, wherein said initiator has a 3′-terminal sequence of 5′-dI-dT-3′.

25. The method of claim 21, wherein said TdT is a TdT variant.

26. The method of claim 19, wherein each of said first labels is selected from the group consisting of fluorescent dyes and fluorescent quenchers such that at least one of said first labels is a fluorescent dye.

27. The method of claim 19, wherein said 3′-O-blocked-(alkyne/azide)-nucleoside triphosphate or said 3′-O-blocked-(protected alkyne/protected azide)-nucleoside triphosphate is a 3′-O-blocked-alkyne-nucleoside triphosphate or a 3′-O-blocked-protected alkyne-nucleoside triphosphate and wherein each of said first labels has an azide group.

28. The method of claim 19, wherein said plurality of labels comprises two labels and said first label is a fluorescent dye and said second label is a fluorescent quencher, and wherein said 3′-O-blocked-alkyne-nucleoside triphosphate or 3′-O-blocked-protected alkyne-nucleoside triphosphate is a 3′-O-blocked-alkyne-nucleoside triphosphate.

29. The method of claim 28, wherein said 3′-O-blocked-alkyne-nucleoside triphosphate is incorporated immediately after said terminal nucleotide of said initiator.

30. The method of claim 19, wherein said 3′-O-blocked-nucleoside triphosphates are 3′-O—NH2-nucleoside triphosphates.

31. The method of claim 19, wherein said 3′ -alternaive blocking group or 3′-O-alternative blocking group is 3′ -azido or 3′-O-azido and said second label is a fluorescent quencher whose said complementary moiety is an alkyne.

32. The method of claim 19, wherein said 3′ -alternaive blocking group or 3′-O-alternative blocking group is 3′-O—NH2 and wherein said second label is a fluorescent quencher whose said complementary moiety is an aldehyde.

33. A kit for enzymatically synthesizing a polynucleotide probe comprising at least one 3′-O-blocked-(alkyne/azide)-nucleoside triphosphate or 3′-O-blocked-(protected alkyne/protected azide)-nucleoside triphosphate and a nucleoside triphosphate having a 3′-alternative blocking group or 3′-O-alternative blocking group, wherein the alternative blocking group has a reactive moiety and may be the same or different than a 3′-O-blocking group of the 3′-O-blocked-(alkyne/azide)-nucleoside triphosphate or 3′-O-blocked-(protected alkyne/protected azide)-nucleoside triphosphate.

34. The kit of claim 33, further comprising at least one first label having an (alkyne/azide) group and a second label having a complementary moiety to said reactive moiety.

35. The kit of claim 33, wherein (i) said at least one 3′-O-blocked-(alkyne/azide)-nucleoside triphosphate or3′-O-blocked-(protected alkyne/protected azide)-nucleoside triphosphate is at least one 3′-O—NH2-alkyne-nucleoside triphosphate, (ii) each of said at least one first labels has an azide group, (iii) said 3′-alternative blocking group or 3′-O-alternative blocking group is 3′-O—NH2, and (iv) said complementary moiety of said second label is an aldehyde group.

36. The kit of claim 33, wherein at least one of said first labels is a fluorescent dye and said second label is a fluorescent quencher capable of quenching fluorescent emissions of the fluorescent dye.
